(19) United States
(12) Patent Application Publication
(10) Pub. No.: US 2017/0265897 A1
(43) Pub. Date: Sep. 21, 2017

(54) DISSECTOR AND PULLER INSTRUMENTS

(71) Applicant: Joseph A. Grocela, Weston, MA (US)

(72) Inventor: Joseph A. Grocela, Weston, MA (US)

(21) Appl. No.: 15/073,755

(22) Filed: Mar. 18, 2016

(51) Int. Cl. A61B 17/50; A61N 1/05; A61B 90/00

(52) U.S. Cl. CPC A61B 17/50; A61B 90/02; A61N 1/0502

(57) ABSTRACT

A kit for removing an embedded wire from tissue includes a dissector instrument for dissecting tissue surrounding an embedded wire and a pulling instrument for pulling the embedded wire from the tissue. The dissector instrument includes a proximal portion with a first and second handle and a distal portion. The distal portion of the dissector instrument includes a tubular section for surrounding an embedded wire. The pulling instrument includes a proximal portion with a first and second handle and a distal portion. The distal portion of the pulling instrument includes an elongated clamp for coupling to the embedded wire for extraction. The dissector instrument and pulling instrument may be used together as a kit, or separately to extract embedded wire from tissue.

### BACKGROUND OF THE INVENTION

1. Field of the Invention

The subject disclosure relates to methods and instruments for removing items from tissue, and more particularly to improved methods and instruments for removing wires, jewelry and like embedded objects with minimal surrounding tissue damage.

2. Background of the Related Art

Wires may be embedded in tissues for a variety of reasons. For example, embedded wires provide treatment for urinary and fecal incontinence. Transcutaneous electrical nerve stimulation (TENS) is another common treatment that can benefit from embedded wires in treating chronic pain. Pacemakers and various coronary devices include embedded wires. For example, see U.S. Pat. No. 7,467,017 issued on Dec. 16, 2008 and U.S. PG Pub. No. 2013/00030512 published on Jan. 31, 2013, which are incorporated herein by reference. In addition to wires, there are various tubes and other appliances, such as catheters, that are embedded. There are also the jewelry and ornamental piercings that have become so popular.

### SUMMARY

The prior art method of removing embedded wires from the body is to dissect the tissue around the wire. After dissection, a clamp is used to grasp the wire. However, the jaws of the clamp tend to crush the wire because the force is applied to a small area. Crushing causes relatively weak spots that can lead to breaking even when the wire is pulled lightly. The subject technology improves dissection and eliminates local crushing of the wire, thus reducing the risk of breakage and/or leaving foreign material in the tissue.

In view of the temporary nature of placement of various wires and the like in tissue, a need exists for instruments and methods for easily and reliably removing the objects with minimal surrounding tissue damage.

Featured, in one embodiment, is an instrument for dissecting tissue surrounding an embedded wire. The dissecting instrument includes a first lever arm having a first proximal handle portion and a first distal portion. The first distal portion includes a first tip coupled to a cylindrical shell having a first semi-circular portion. A second lever arm has a second proximal handle portion and a second distal portion. The second distal portion includes a second tip coupled to a second semi-circular portion. A pin pivotally connects the first and second lever arms so that when the lever arms are closed, the second semi-circular portion mates with the first semi-circular portion to complete the cylindrical shell. In one design, the cylindrical shell forms an arcuate shape. In another design, the first lower end of the semi-circular portion and a second lower end of the second semi-circular portion are beveled for insertion into the tissue.

The subject technology is also directed to an instrument for dissecting tissue surrounding an embedded item having a first lever arm with a first distal tip coupled to a first shell having a first portion for sliding over the item. The first lever arm also has a second portion for contacting a portion of the item. A second lever arm hingedly connects to the first lever arm. The second lever arm has a second distal tip coupled to a second shell complimentary to the first shell so that when the lever arms are closed, an inner portion of the shells matches a profile of the item.

Also featured in one embodiment is an instrument for pulling an embedded wire from tissue. The puller instrument includes a first lever arm having a first proximal handle portion and a first distal portion. The first distal portion includes a first tip coupled to a cylindrical shell having a first semi-circular portion. A second lever arm has a second proximal handle portion and a second distal portion. The second distal portion includes a second tip coupled to a second semi-circular portion. A pin pivotally connects the first and second lever arms so that when the lever arms are closed, the second semi-circular portion can capture the embedded wire against the first semi-circular portion for reliably pulling the wire.

Also featured in one embodiment is a kit for removing a wire embedded in tissue comprising a dissector instrument for dissecting tissue surrounding an embedded wire and a pulling instrument for pulling the embedded wire from the tissue. The dissector instrument has a tubular section for surrounding the embedded wire during extraction. The pulling instrument has an elongated clamp for coupling to the embedded wire for extraction. In one design the dissector instrument includes a first lever arm having a first proximal handle portion and a first distal portion. The first distal portion forms a first tip coupled to a cylindrical shell having a first semi-circular portion, wherein the first semi-circular portion forms part of the tubular section. A second lever arm has a second proximal handle portion and a second distal portion. The second distal portion forms a second tip coupled to a second semi-circular portion, wherein the second semi-circular portion forms part of the tubular section. A pin pivotally connects the first and second lever arms so that when the lever arms are closed, the second semi-circular portion mates with the first semi-circular portion to complete the cylindrical shell. In one design, the pulling instrument includes a first lever arm having a first proximal handle portion and a first distal portion. The first distal portion forms a first tip coupled to a cylindrical shell having a first semi-circular portion and the first semi-circular portion forms part of the elongated clamp. A second lever arm has a second proximal handle portion and a second distal portion. The second distal portion forms a second tip coupled to a second semi-circular portion and the second semi-circular portion forms part of the elongated clamp. A pin pivotally connects the first and second lever arms so that when the lever arms are closed, the second semi-circular portion can capture the embedded wire against the first semi-circular portion for reliably pulling the wire.

It should be appreciated that the subject technology can be implemented and utilized in numerous ways, including without limitation as a process, an apparatus, a system, a device, a method for applications now known and later developed. These and other unique features of the system disclosed herein will become more readily apparent from the following description and the accompanying drawings.

### BRIEF DESCRIPTION OF THE DRAWINGS

So that those having ordinary skill in the art to which the disclosed system appertains will more readily understand how to make and use the same, reference may be had to the following drawings.

FIG. 1 is a perspective view of a dissector instrument being readied to remove a wire from tissue in accordance with the subject disclosure.

FIG. 2 is a perspective view of a dissector instrument dissecting a wire from tissue in accordance with the subject disclosure.

FIG. 3 is an enlarged detailed view of the area circled in FIG. 2.

FIG. 4 is a cross-sectional view of the dissector instrument being inserted in tissue in the closed position in accordance with the subject disclosure.

FIG. 5 is a cross-sectional view of the dissector instrument separating tissue from a wire in accordance with the subject disclosure.

FIG. 6 is a cross-sectional view of the dissector instrument further separating tissue from a wire having tines for retaining the wire in place in accordance with the subject disclosure.

FIG. 7 is a cross-sectional view of the dissector instrument covering the tines from a wire for removal in accordance with the subject disclosure.

FIG. 8 is a perspective view of a puller instrument being readied to remove a wire from tissue in accordance with the subject disclosure.

FIG. 9 is a perspective view of a puller instrument closed on wire for removal from tissue in accordance with the subject disclosure.

FIG. 10 is a cross-sectional view along line 10-10 of FIG. 9.

### DETAILED DESCRIPTION OF PREFERRED EMBODIMENTS

The subject technology improves upon the prior art associated with removing embedded objects from tissue. The advantages, and other features of the instruments and methods disclosed herein, will become more readily apparent to those having ordinary skill in the art from the following detailed description of certain preferred embodiments taken in conjunction with the drawings which set forth representative embodiments of the present invention. It is understood that references to the figures such as lower, upper, up, down, upward, downward, left, and right are with respect to the figures and not meant in a limiting sense.

### The Dissector Instrument

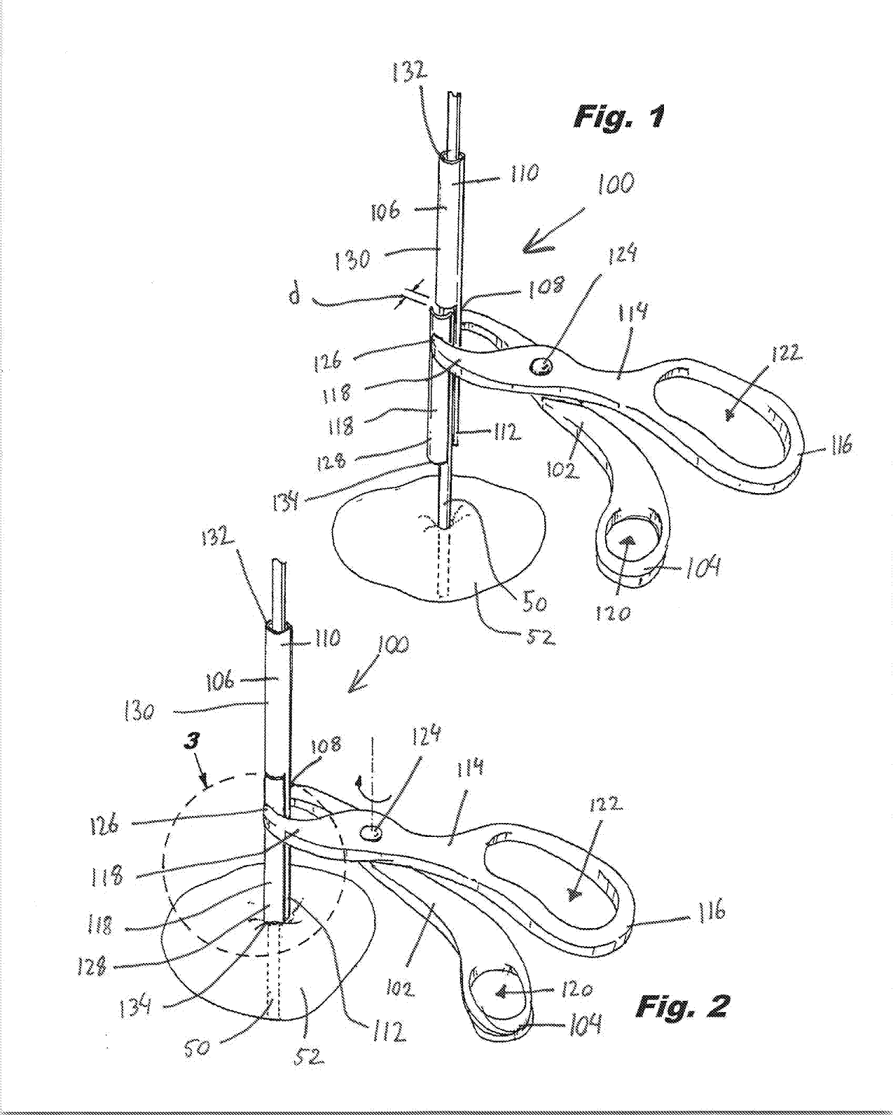

Referring now to FIGS. 1 and 2, perspective views of a dissector instrument 100 readied to remove a wire 50 from tissue 52 and dissecting tissue 52 from wire 50 in accordance with the subject disclosure are shown. The dissector instrument 100 includes a first lever arm 102 having a first proximal handle portion 104 and a first distal portion 106. The first distal portion 106 forms a first tip 108 coupled to a cylindrical shell 110 having a first semi-circular portion 112. The shell 110 may be any shape such as oval, triangular, rectangular and the like in order to match the profile of the embedded item. The shell 110 may extend transverse to the lever arm 102 as shown, parallel or at any angle in between depending upon the particular application for which the dissector instrument 100 is designed.

A second lever arm 114 has a second proximal handle portion 116 and a second distal portion 118. The first and second proximal handle portions 104,116 form openings 120, 122 for the fingers and thumb so that the dissector instrument 100 can be used with a single hand. A pin 124 pivotally connects the first and second lever arms 102,114 together.

The second distal portion 118 also forms a second tip 126 coupled to a second semi-circular portion 128. The second semi-circular portion 128 is complimentary to the first semi-circular portion 112 so that when the lever arms 102, 114 are closed, the second semi-circular portion 128 completes the first semi-circular portion 112. In other words, when the tips 108, 126 are squeezed together, the distal portions roughly form a cylinder 130 with an upper end 132 and a lower end 134. The inner diameter “d” of the cylinder 130 is approximately equal to the diameter of the wire 50 but slightly larger so that the cylinder 130 can be moved along the embedded wire 50 when closed.

Still referring to FIGS. 1 and 2, in operation, the first distal portion 106 is slid onto the wire 50 and the lever arms 102, 114 are closed to complete the cylinder 130. Because the inner diameter of the cylinder 130 is slightly larger than the wire 50, the cylinder 130 easily moves along the wire 50. The lower end 134 is adjacent the tissue 52 to contact the tissue 52 first as the cylinder 130 slides down the wire 50.

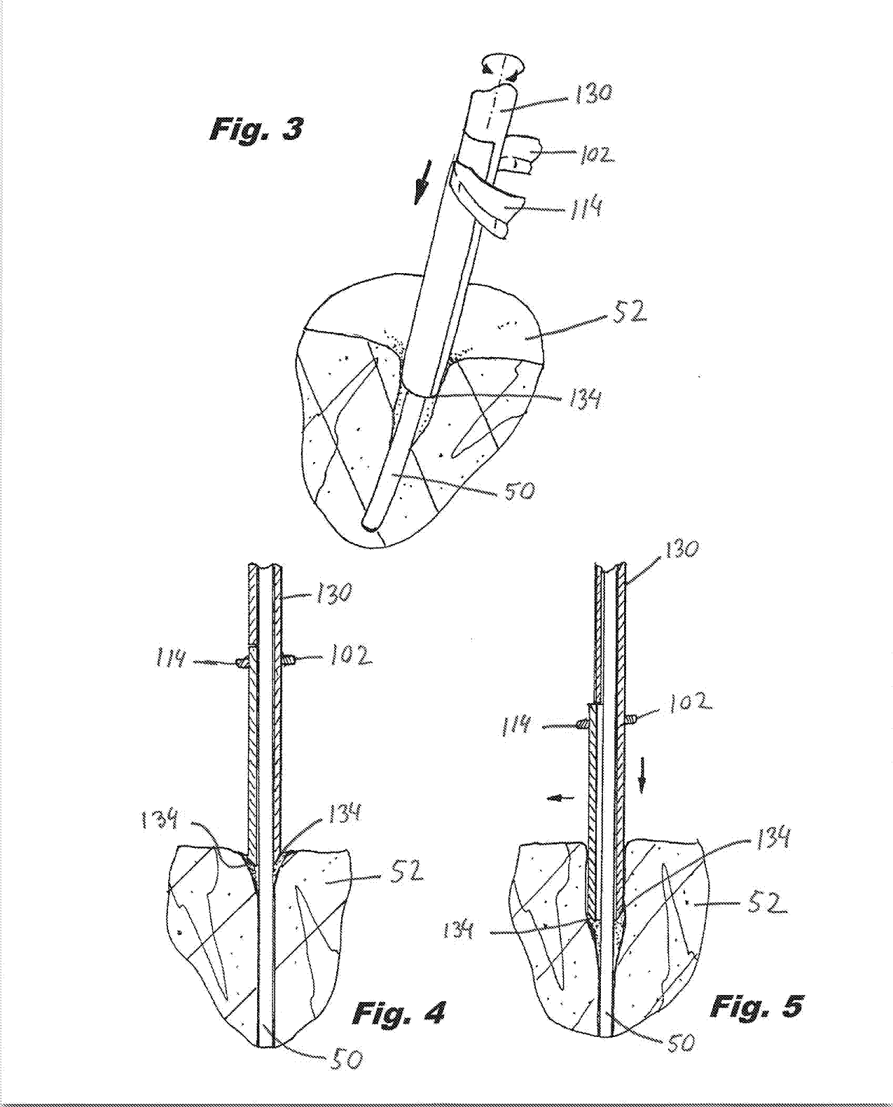

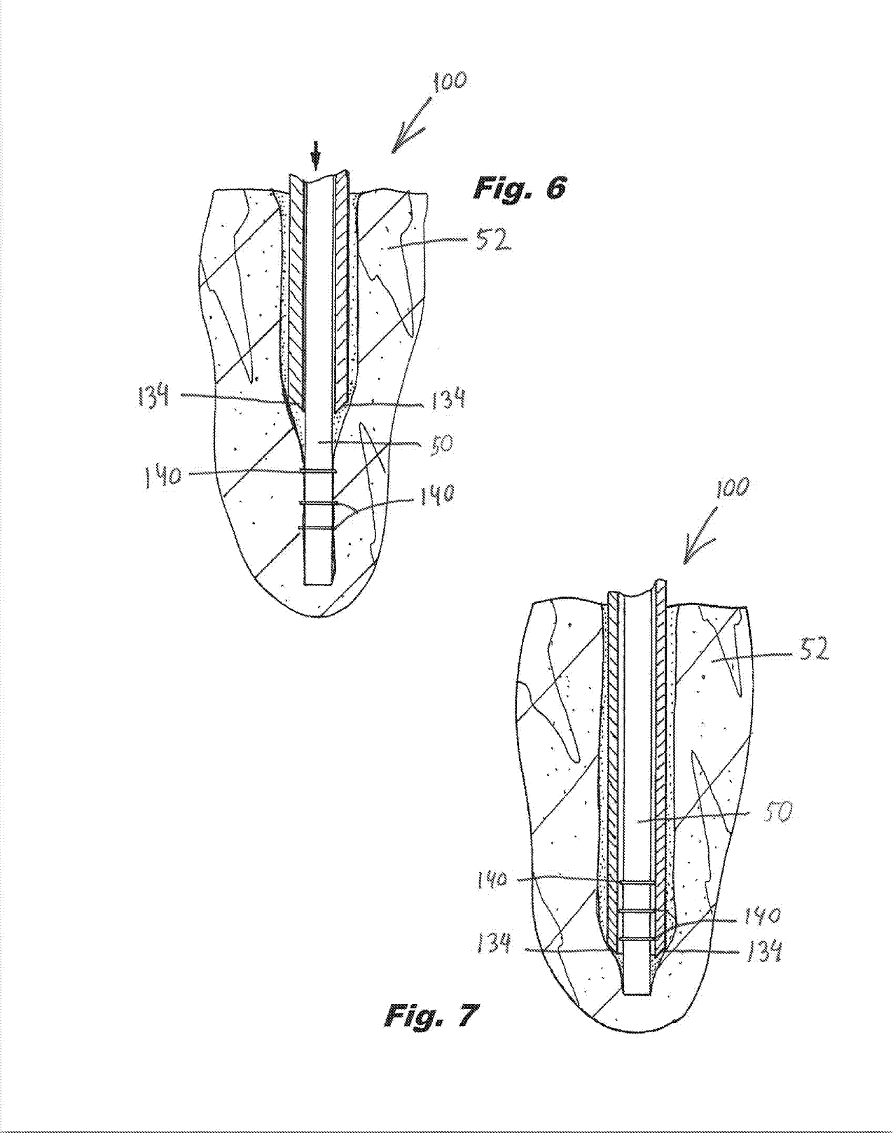

Referring now to FIG. 3, an enlarged detailed view of the area circled in FIG. 2 is shown with the tissue 52 drawn in cross-section. The lower end 134 of the cylinder 130 is beveled (best shown in FIG. 6) so that as the lower end 134 slides down the wire 50, the lower end 134 comes between the wire 50 and the tissue 52. The lower end 134 is worked into the tissue 52 by gentle pressure and/or rotation as shown in cross-sectional view in FIGS. 4 and 5. To additionally facilitate dissection of the tissue 52 from the wire 50, once the lower end 134 is within the tissue 52, the lever arms 102, 114 may be opened, closed and rotated to separate the tissue 52 from the wire 50 circumferentially. The surgeon continues the process of inserting, rotating and opening the lower end 134 until sufficient tissue is separated for removal of the wire 50.

Referring now to FIGS. 6 and 7, cross-sectional views of the dissector instrument 100 further separating tissue 52 from a wire 50 having tines 140 is shown. The tines 140 help retain the wire 50 in place. The dissection procedure is similar to the one described above. The lower end 134 is worked into the tissue until the lower 134 end reaches the tines 140. At this point, the surgeon continues opening, closing, rotating and inserting the lower end 134 to separate the tissue 52 from the tines 140. Eventually, the lower end 134 surrounds the tines 140 as shown in FIG. 7, which indicates that the tissue 52 has been separated from the wire 50 and tines 140 to be ready for removal. As such, the wire 50 can be extracted while the dissector 100 is in place.

### The Puller Instrument

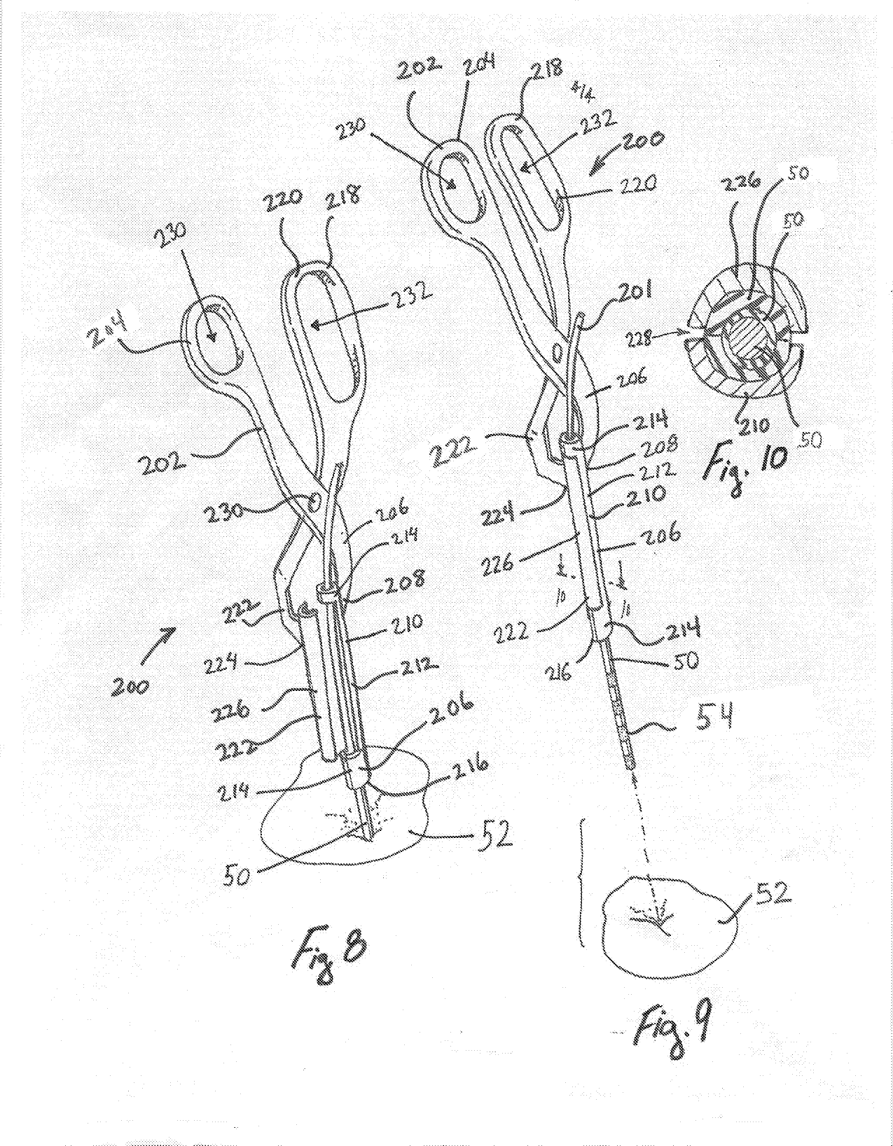

Referring now to FIGS. 8-10, a puller instrument 200 for removing an embedded wire 50 after dissection is shown. The puller instrument 200 includes a first lever arm 202 having a first proximal handle portion 204 and a first distal portion 206. The first distal portion 206 forms a first tip 208 coupled to a cylindrical shell 210. The cylindrical shell 210 has a first central semi-circular portion 212 with fixed collars 214 on each end. It is envisioned that the lower end 216 is beveled for insertion into tissue 52 and may be any length needed to accomplish the function described herein.

A second lever arm 218 has a second proximal handle portion 220 and a second distal portion 222. The first and second proximal handle portions 204, 220 form openings 230, 232 for the fingers and thumb so that the puller instrument 200 can be used with a single hand. The second distal portion 222 forms a second tip 224 coupled to a second semi-circular portion 226. A pin 230 pivotally connects the first and second lever arms 202, 218 so that when the lever arms 202, 218 are closed, the second semi-circular portion 226 can capture a wire 50 against the first semi-circular portion 212 for reliably pulling the wire 50. To reliably capture wire 50 against first semi-circular portion 212, second semi-circular portion 226 may form a smaller diameter than wire 50, form a small arc, or the like.

As would be appreciated by one of ordinary skill in the art based upon review of the subject disclosure, the dissector instrument 100 and the puller instrument 200 can be used consecutively to dissect tissue from an embedded wire, then remove the wire. In operation, the fixed collars 214 of the puller instrument 200 are threaded onto the wire 50 as shown in FIG. 8. The lower end 216 is worked into the dissected tissue and preferably covers the tines if present. Then, the puller instrument 200 is closed so that the second semi-circular portion 226 captures the wire 50 against the first semi-circular portion 212. The wire 50 is shown as having electrodes 54. For example, as shown in FIG. 10, the inner diameter of the cylindrical shell 210 is slightly less than the outer diameter of the wire 50, creating axial gaps 228 and allowing wire 50 to be securely gripped with a minimal likelihood of damage. Once the wire 50 is securely gripped, the puller instrument 200 draws out the wire 50 as shown in FIG. 9.

In another approach, the dissector instrument 100 and the puller instrument 200 are used simultaneously. The dissector instrument 100 is used to dissect the tissue and free up the wire. While holding the dissection instrument 100 closed in place around the wire, the puller instrument 200 is coupled to the wire and used for extraction.

In another embodiment, the dissector instrument is adapted to perform dissection and removal. The semi-circular portions are sized to grip the wire. As such, the dissection is performed similarly to that described. The surgeon can work the dissector instrument along the wire into the tissue provided that the dissector instrument is not closed enough to grip the wire. Once the desired dissection and insertion depth are reached, the dissector instrument can be closed to grip and extract the wire.

It will be appreciated by those of ordinary skill in the pertinent art that the functions of several elements may, in alternative embodiments, be carried out by fewer elements, or a single element. Similarly, in some embodiments, any functional element may perform fewer, or different, operations than those described with respect to the illustrated embodiment. Also, functional elements (e.g., tip, shells, handles and the like) shown as distinct for purposes of illustration may be incorporated within other functional elements in a particular implementation. Additionally, the size and shape of the instruments can not only be adapted to the size and shape of the wire, but can be modified for removal of any item (e.g., oddly shaped jewelry) from tissue.

While the subject technology has been described with respect to preferred embodiments, those skilled in the art will readily appreciate that various changes and/or modifications can be made to the subject technology without departing from the spirit or scope of the subject technology as defined by the claims.

What is claimed is:

1. An instrument for dissecting tissue surrounding an embedded wire, the instrument comprising:
a first lever arm having a first proximal handle portion and a first distal portion, wherein the first distal portion includes a first tip coupled to a cylindrical shell having a first semi-circular portion;
a second lever arm having a second proximal handle portion and a second distal portion, wherein the second distal portion includes a second tip coupled to a second semi-circular portion; and
a pin pivotally connecting the first and second lever arms so that when the lever arms are closed, the second semi-circular portion mates with the first semi-circular portion to complete the cylindrical shell.

2. An instrument as recited in claim 1, wherein the cylindrical shell forms an arcuate shape.

3. An instrument as recited in claim 1, wherein a first lower end of the semi-circular portion and a second lower end of the second semi-circular portion are beveled for insertion into the tissue.

4. An instrument for dissecting tissue surrounding an embedded item, the instrument comprising:
a first lever arm having a first distal tip coupled to a first shell having a first portion for sliding over the item and a second portion for contacting a portion of the item; and
a second lever arm hingedly connected to the first lever arm, the second lever arm having a second distal tip coupled to a second shell complimentary to the first shell so that when the lever arms are closed, an inner portion of the shells matches a profile of the item.

5. An instrument for pulling an embedded wire from tissue, the instrument comprising:
a first lever arm having a first proximal handle portion and a first distal portion, wherein the first distal portion forms a first tip coupled to a cylindrical shell having a first semi-circular portion;
a second lever arm having a second proximal handle portion and a second distal portion, wherein the second distal portion forms a second tip coupled to a second semi-circular portion; and
a pin pivotally connecting the first and second lever arms so that when the lever arms are closed, the second semi-circular portion can capture the embedded wire against the first semi-circular portion for reliably pulling the wire.

6. An instrument as recited in claim 1, wherein a lower end of the cylindrical shell is beveled for easy insertion into the tissue.

7. A kit for removing a wire embedded in tissue comprising:
a dissector instrument for dissecting tissue surrounding an embedded wire, the dissector instrument having tubular section for surrounding the embedded wire during extraction; and
a pulling instrument for pulling the embedded wire from the tissue, the pulling instrument having an elongated clamp for coupling to the embedded wire for extraction.

8. A kit as recited in claim 7, wherein the dissector instrument comprises:
a first lever arm having a first proximal handle portion and a first distal portion, wherein the first distal portion forms a first tip coupled to a cylindrical shell having a first semi-circular portion, wherein the first semi-circular portion forms part of the tubular section;
a second lever arm having a second proximal handle portion and a second distal portion, wherein the second distal portion forms a second tip coupled to a second semi-circular portion, wherein the second semi-circular portion forms part of the tubular section; and
a pin pivotally connecting the first and second lever arms so that when the lever arms are closed, the second semi-circular portion mates with the first semi-circular portion to complete the cylindrical shell.

9. A kit as recited in claim 7, wherein the pulling instrument comprises:
a first lever arm having a first proximal handle portion and a first distal portion, wherein the first distal portion forms a first tip coupled to a cylindrical shell having a first semi-circular portion and the first semi-circular portion forms part of the elongated clamp;
a second lever arm having a second proximal handle portion and a second distal portion, wherein the second distal portion forms a second tip coupled to a second semi-circular portion and the second semi-circular portion forms part of the elongated clamp; and
a pin pivotally connecting the first and second lever arms so that when the lever arms are closed, the second semi-circular portion can capture the embedded wire against the first semi-circular portion for reliably pulling the wire.
